(19) United States
(12) Patent Application Publication
(10) Pub. No.: US 2009/0239238 A1
(43) Pub. Date: Sep. 24, 2009

(54) METHODS FOR MEASURING PRO-INFLAMMATORY SUBSTANCE LEVELS IN DIALYSIS SOLUTIONS AND DIALYSIS COMPONENTS

(75) Inventors: Run Wang, Gurnee, IL (US); Cliff Holmes, Glenview, IL (US); Line Skoufos, Mt. Prospect, IL (US); Pat Hyla, Lake Zurich, IL (US)

Correspondence Address: K&L Gates LLP, P.O. Box 1135, Chicago, IL, 60690-1135, US

(73) Assignees: BAXTER INTERNATIONAL INC., Deerfield, IL (US); BAXTER HEALTHCARE S.A., Zurich (CH)

(21) Appl. No.: 12/052,477

(22) Filed: Mar. 20, 2008

(51) Int. Cl. G01N 33/53

(52) U.S. Cl. 435/7.1

(57) ABSTRACT

Methods of measuring levels of pro-inflammatory substances in dialysis solutions or specific dialysis components used to manufacture dialysis solutions are provided. In a general embodiment, the method comprises determining an IL-6 response versus a pro-inflammatory substance concentration of a reference standard containing a pro-inflammatory substance and establishing a dose-response curve of the IL-6 responses as a function of different pro-inflammatory substance concentrations of the reference standard. An IL-6 response of a test sample of a dialysis solution is determined. The corresponding pro-inflammatory substance concentration of the dialysis solution is then calculated using the dose-response curve. The IL-6 response of the reference standards and the dialysis solution can be determined using a high sensitivity PBMC IL-6 assay.

### BACKGROUND

The present disclosure relates generally to dialysis solutions. More particularly, the present disclosure relates to methods of measuring pro-inflammatory substances in dialysis solutions or dialysis components used for manufacturing dialysis solutions.

Parenteral pharmaceutical products are required to be free of contaminating substances, such as those that might cause peritonitis. Peritonitis, or inflammation of the peritoneum, is a major complication of peritoneal dialysis. Peritonitis may be caused by intraperitoneal bacterial infections. Alternatively, peritonitis caused by a chemical or a foreign body irritant is known as aseptic or sterile peritonitis. Despite existing testing of peritoneal dialysis solutions, outbreaks of aseptic peritonitis still occur.

### SUMMARY

The present disclosure generally relates to methods of measuring levels of pro-inflammatory substances in dialysis solutions or dialysis components (i.e. raw materials used to manufacture dialysis solutions). In a general embodiment, the method comprises determining an IL-6 response versus a pro-inflammatory substance concentration of a reference standard containing the pro-inflammatory substance and establishing a dose-response curve of the IL-6 response as a function of two or more different pro-inflammatory substance concentrations of the reference standard. An IL-6 response of the dialysis solution is determined. The corresponding pro-inflammatory substance concentration of the dialysis solution is then calculated using the dose-response curve. The IL-6 response of the reference standards and the dialysis solution can be determined using a high sensitivity Peripheral Blood Mononuclear Cell (PBMC) IL-6 assay.

In an embodiment, the dose-response curve of the IL-6 response is established as a function of three or more different pro-inflammatory substance concentrations of the reference standard. The pro-inflammatory substance concentrations can cover a pro-inflammatory substance range of two, three, four or more orders of magnitude.

In an embodiment, the tested solution can be a dialysis solution. The dialysis solution can comprise a glucose polymer or glucose polymer derivative as an osmotic agent. The glucose polymer can be icodextrin.

In another embodiment, the present disclosure provides a method for measuring peptidoglycan in a dialysis solution. The method comprises determining an IL-6 response versus a peptidoglycan concentration of a reference standard containing the peptidoglycan and establishing a dose-response curve of the IL-6 response as a function of at least two different peptidoglycan concentrations of the reference standard. An IL-6 response of the dialysis solution can be determined. The corresponding peptidoglycan concentration of the dialysis solution can then be calculated using the dose-response curve.

In an alternative embodiment, the present disclosure provides a method of measuring an inflammatory substance in a glucose polymer or a glucose polymer derivative. The method comprises determining an IL-6 response versus a pro-inflammatory substance concentration of a reference standard containing the pro-inflammatory substance and establishing a dose-response curve of the IL-6 response as a function of two or more different pro-inflammatory substance concentrations of the reference standard. An IL-6 response of the glucose polymer or glucose polymer derivative is determined. The corresponding pro-inflammatory substance concentration of the, glucose polymer or glucose polymer derivative is then calculated using the dose-response curve. The IL-6 response of the reference standards and the glucose polymer or glucose polymer derivative can be determined using a high sensitivity PBMC IL-6 assay.

In yet another embodiment, the present disclosure provides methods for manufacturing a dialysis solution. The method can include any suitable number and type of processing stages. For example, the method can comprise providing one or more dialysis components such as a glucose polymer or a glucose polymer derivative, measuring the pro-inflammatory substance concentration of the dialysis component using a high sensitivity PBMC IL-6 assay and preparing a peritoneal dialysis solution using the dialysis component if the pro-inflammatory substance concentration of the dialysis component is below a threshold concentration.

In another embodiment, the present disclosure provides a method of providing dialysis to a patient. The method comprises determining an IL-6 response versus a pro-inflammatory substance concentration of a reference standard containing the pro-inflammatory substance and establishing a dose-response curve of the IL-6 response as a function of two or more different pro-inflammatory substance concentrations of the reference standard. An IL-6 response of the dialysis solution is determined. The corresponding pro-inflammatory substance concentration of the dialysis solution is then calculated using the dose-response curve. The dialysis solution can then be administered to the patient. For example, the dialysis solution can be administered to the patient only if the pro-inflammatory substance concentration of the dialysis solution is below a threshold level that can be established.

An advantage of the present disclosure is to provide improved methods of determining the pro-inflammatory characteristics of a dialysis solution.

Another advantage of the present disclosure is to provide an improved method for measuring peptidoglycan in an solution.

Yet another advantage of the present disclosure is to provide improved dialysis solutions.

Still another advantage of the present disclosure is to provide improved testing procedures that can be employed to prevent peritonitis in patients that receive peritoneal dialysis therapy.

Another advantage is of the present disclosure is to provide improved methods for manufacturing and using dialysis solutions that employ a detection protocol to determine the presence of peptidoglycan in the dialysis solution.

Additional features and advantages are described herein, and will be apparent from the following Detailed Description and the figures.

### BRIEF DESCRIPTION OF THE FIGURES

FIG. 1 is a graph showing a dose-response curve of IL-6 response (pg/mL) versus peptidoglycan concentration (ng/mL).

FIGS. 2A and 2B are graphs showing the effect of increasing cell concentrations on the IL-6 response using the PBMC IL-6 assay.

FIG. 3 is a graph showing IL-6 responses from a sample donor.

FIG. 4 is a graph showing IL-6 responses of icodextrin raw material.

FIG. 5 is a graph showing a dose-response curve generated with a PBMC IL-6 versus a dose-response curve generated using a high sensitivity PBMC IL-6 assay.

FIG. 6 is a graph showing a comparison of the IL-6 response of a contaminated dialysis solution as determined using a dose-response curve from a control contaminated dialysis solution using a high sensitivity PBMC IL-6 assay.

FIG. 7 is a graph showing the capability of the PBMC IL-6 assay to discriminate between contaminated and non-contaminated dialysis solutions based on a geometric median.

FIG. 8 is a graph showing the IL-6 response versus the peptidoglycan concentration determined for icodextrin raw material samples.

FIG. 9 is a graph showing the capability of the PBMC IL-6 assay to discriminate between contaminated and non-contaminated icodextrin raw material based on a geometric median.

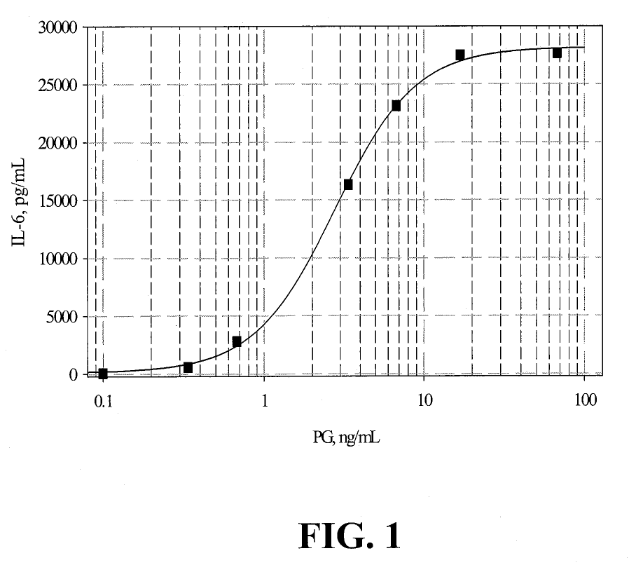

### DETAILED DESCRIPTION

The present disclosure generally relates to methods of measuring levels of pro-inflammatory substances in a dialysis solution or dialysis components (i.e. raw materials used to manufacture dialysis solutions). The pro-inflammatory substance can be, for example, peptidoglycan. Peptidoglycan is a major component of a gram positive bacterial cell wall and thus can serve as a marker for gram positive bacteria. In this regard, testing for pro-inflammatory substances such as peptidoglycan can be utilized to effectively prevent peritonitis in patients that use dialysis solutions, for example, such as peritoneal dialysis solutions that contain a glucose polymer such as icodextrin and the like.

Icodextrin is derived from corn starch, a natural product. It is well known that products of natural origin are contaminated with a wide variety of micro-organisms. The inventors have found that some natural products, such as corn starch, contain an acidophilic thermophilic bacteria, such as Alicyclobacillus acidocaldarius. The later organism is ubiquitous in the food industry, particularly in acidic beverages. It is the alicyclobacillus that produces guaiacol, which is a causative substance for an “off” flavor orange juice.

Aseptic peritonitis associated with icodextrin-based peritoneal dialysis solutions is believed to be the largest adverse event reported for a peritoneal dialysis solution due to a contaminant of microbial origin. Based on the experimental investigations, peptidoglycan in the glucose polymer or glucose polymer derivative-based peritoneal dialysis solution may be a causative agent of aseptic peritonitis. Further, pharmacovigilence data from previous studies supports the effectiveness of a corrective action and manufacturing screening procedure to prevent the occurrence of peritonitis. These findings illustrate that while endotoxin is deservedly one of the more worrisome bacterial product that can cause adverse effects to patients, it is not the sole one. In this regard, non-endotoxin pyrogens, such as peptidoglycans, are capable of producing clinically significant inflammation. Thus, this demonstrates that parenteral pharmaceutical products that pass the compendial tests and so meet Pharmacopoeia standards may still require a further level of testing to effectively determine the efficacy and safe use of such products to better ensure quality of life issues associated with use of same.

In a general embodiment, the present disclosure provides a method of measuring or quantifying levels of one or more pro-inflammatory substances in a dialysis solution. The method comprises determining an IL-6 response versus a pro-inflammatory substance concentration of a reference standard containing the pro-inflammatory substance. A reference standard with known pro-inflammatory substance concentrations can be used.

The IL-6 response caused by the pro-inflammatory substance can be determined using a high sensitivity Peripheral Blood Mononuclear Cell (PBMC) IL-6 release assay (the “PBMC IL-6 assay”). For example, the pro-inflammatory substance can be measured in the reference standards from a test that utilizes cytokine secreted from stimulated mononuclear cells. In embodiments of the present disclosure, the PBMC IL-6 assay can be made more sensitive by 1) using an increased cell concentration (e.g. 2 million cells/mL or higher, preferably 4 million cells/mL ), 2) using the highest purity cells and/or 3) using cells taken only from blood donors not taking any medication with known anti-inflammatory properties.

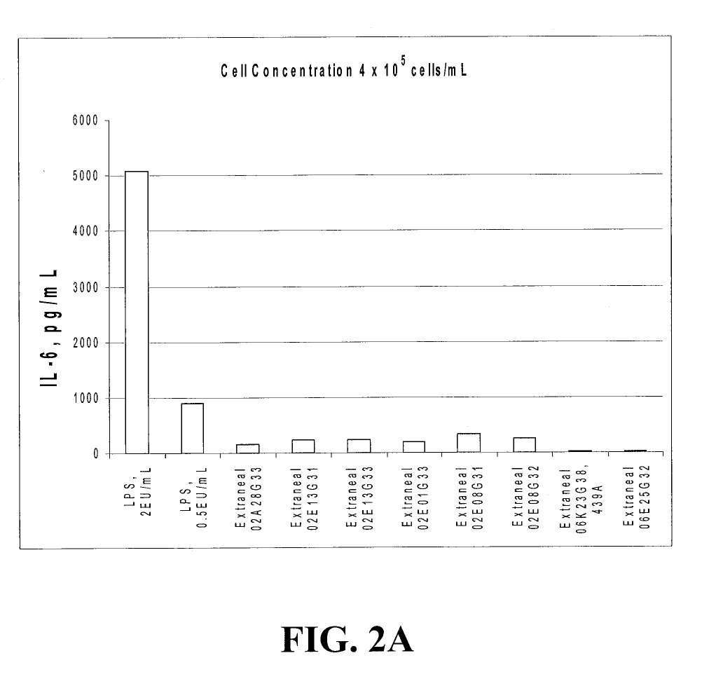

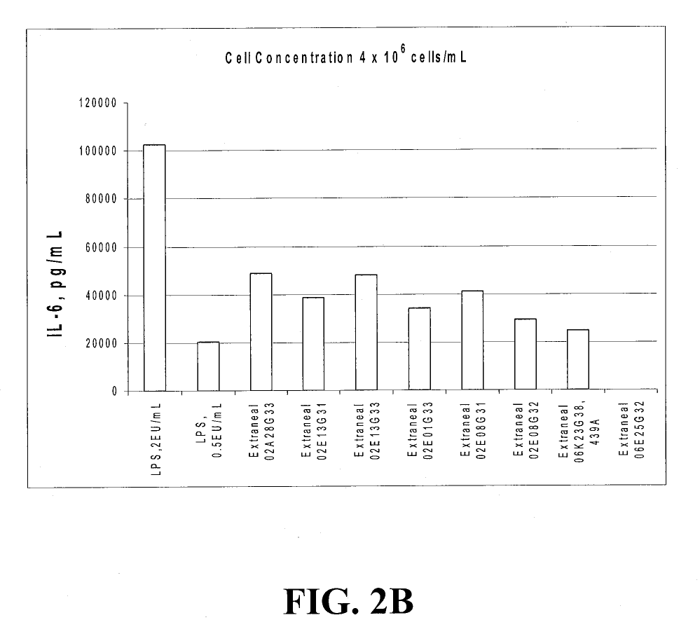

As shown in FIG. 2, a dose-response curve can be established using the IL-6 response versus the corresponding pro-inflammatory substance concentration (e.g. peptidoglycan) of the reference standard. A data point is plotted on the graph for every IL-6 response and its corresponding pro-inflammatory substance concentration that caused the IL-6 response. The different pro-inflammatory substance concentrations can be made by diluting an initial known concentration of the pro-inflammatory substance of the reference standard to desired diluted concentrations to obtain various pro-inflammatory substance concentrations for the dose-response curve.

An IL-6 response of the dialysis solution or test sample of the dialysis solution can be determined using the high sensitivity PBMC IL-6 assay. The corresponding pro-inflammatory substance concentration of the dialysis solution is then calculated using the dose-response curve. In other words, the pro-inflammatory substance concentration in the dialysis solution can be determined using the IL-6 signal from the dialysis solution in combination with the dose-response curve.

In an embodiment, the dose-response curve of the IL-6 response is established as a function of at least three or more different pro-inflammatory substance concentrations of the reference standard. By using at least three points in the dose-response curve, one can establish an effective relationship by targeting the three points at the slope area or between the baseline point and saturated points where the change occurs. At a bare minimum, the three points will comprise one in the middle, one at the baseline and one at the saturated level

In another embodiment, the dose-response curve of the IL-6 response is established as a function of at least seven or more different pro-inflammatory substance concentrations of the reference standard. It should be appreciated that a more accurate dose-response curve can be developed by using as many IL-6 /pro-inflammatory substance data points as possible over a varying pro-inflammatory substance concentration range. The pro-inflammatory substance concentrations can cover a pro-inflammatory substance range of two, three, four or more orders of magnitude.

In an embodiment, the tested solution can be a peritoneal dialysis solution comprising any suitable osmotic agents. In another embodiment, the peritoneal dialysis solution can comprise any one or more suitable glucose polymers or glucose polymer derivatives as an osmotic agent. The glucose polymer can be icodextrin.

In an alternative embodiment, the present disclosure provides a method of measuring an inflammatory substance in dialysis components such as glucose polymers and/or glucose polymer derivatives. The method comprises determining an IL-6 response versus a pro-inflammatory substance concentration of a reference standard containing the pro-inflammatory substance and establishing a dose-response curve of the IL-6 response as a function of two or more different pro-inflammatory substance concentrations of the reference standard. An IL-6 response of the glucose polymers and/or glucose polymer derivatives material is determined. The corresponding pro-inflammatory substance concentration of the glucose polymers and/or glucose polymer derivatives is then calculated using the dose-response curve. The IL-6 response of the reference standards and the glucose polymers and/or glucose polymer derivatives can be determined using a PBMC IL-6 assay. The dialysis components can also be any dialysis ingredient that may comprise an inflammatory substance.

In yet another embodiment, the present disclosure provides methods for manufacturing a dialysis solution. The method can include any suitable number and type of processing stages. For example, the method can comprise providing one or more dialysis components such as glucose polymers or glucose polymer derivatives, measuring the pro-inflammatory substance concentration of the dialysis component using a high sensitivity PBMC IL-6 assay and preparing a peritoneal dialysis solution using the dialysis component if the pro-inflammatory substance concentration of the dialysis component is below a threshold concentration.

If it is determined that the IL-6 response is not in accordance with predetermined criteria, the dialysis component can be further processed to remove the contaminant or to achieve a sufficiently low level of the contaminant. The dialysis component can be further processed in any suitable manner. In an embodiment, the dialysis component can be processed with any suitable number and type of separation devices, such as affinity columns with resins that specifically bind peptidoglycan and/or the like, to make the dialysis component suitable for using in dialysis solutions.

The dialysis solutions can be specifically formulated and suitable for peritoneal dialysis, hemodialysis or any other dialysis therapies. The dialysis solutions can be used, for example, as a single dialysis solution in a single container or as a dialysis part of a separately housed or multi-chambered container. The dialysis solutions can be sterilized using any suitable sterilizing technique such as, for example, autoclave, steam, ultra-violet, high pressure, filtration or combination thereof.

The method of manufacturing a dialysis solution in accordance with the present disclosure can also be used in conjunction with other suitable glucose polymer, glucose polymer derivative or dialysis solution testing procedures. Illustrative examples of suitable testing procedures can be found in U.S. Pat. No. 7,118,857, entitled METHODS AND COMPOSITIONS FOR DETECTION OF MICROBIAL CONTAMINANTS IN PERITONEAL DIALYSIS SOLUTIONS, issued on Oct. 10, 2006, the disclosure of which is herein incorporated by reference. For example, such testing procedures can be generally used to test dialysis glucose polymers and/or glucose polymer derivatives or dialysis solutions. The high sensitivity PBMC IL-6 assay of the present disclosure can be used to further measure whether the glucose polymer/glucose polymer derivative or dialysis solutions contain threshold levels of pro-inflammatory substances such as peptidoglycan.

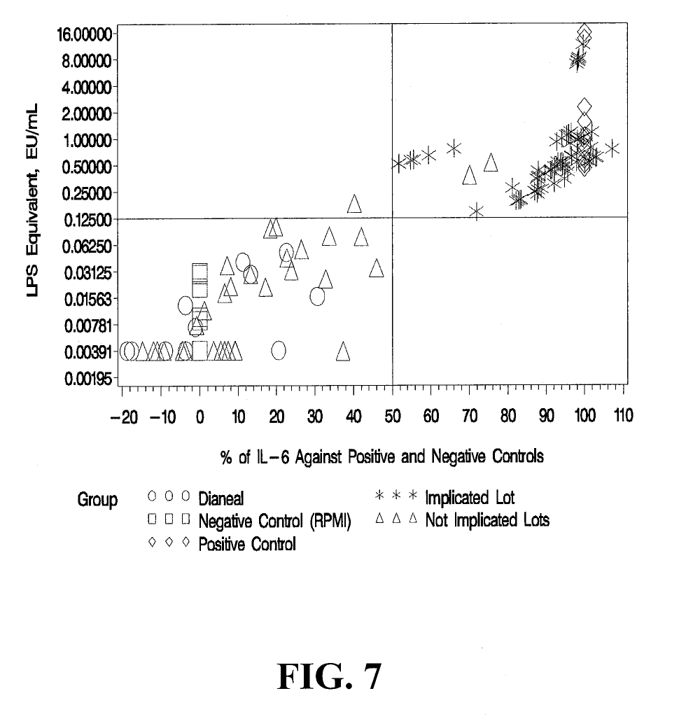

In another embodiment, the present disclosure provides a method of providing a dialysis therapy or treatment to a patient. The dialysis solution can be tested for its ability to cause peritonitis in the patient. The method comprises determining an IL-6 response versus a pro-inflammatory substance concentration of a reference standard containing the pro-inflammatory substance and establishing a dose-response curve of the IL-6 response as a function of two or more different pro-inflammatory substance concentrations of the reference standard. An IL-6 response of the dialysis solution is determined. The corresponding pro-inflammatory substance concentration of the dialysis solution is then calculated using the dose-response curve. The dialysis solution can then be administered to the patient. For example, the dialysis solution can be administered to the patient only if the pro-inflammatory substance concentration of the dialysis solution is below a threshold level that can be established. An example is shown in FIG. 7 where all the peritoneal dialysis solutions along with the negative control samples fall below a response of an endotoxin equivalence of 0.125 EU/mL LPS (lipopolysaccharide).

Ready-to-use formulations of dialysis solutions can be prepared in a number of suitable ways. For example, the dialysis solution can comprise first and second dialysis parts that can be separately stored from each other, such as in separate and hydraulically connected chambers of a multi-chamber container, until mixed together to form a mixed solution. In this regard, the ready-to-use formulation can be prepared within a multiple chamber container by mixing its separate dialysis parts within one chamber of the container. This can effectively eliminate the need to manually inject all or at least a portion of the dialysis parts into the container to form the mixed solution, thus ensuring that the ready-to-use formulation can be readily prepared under sterile conditions.

Further, the multiple chamber container can be configured such that one of the dialysis parts can be placed in direct fluid communication with the patient prior to mixing while the other dialysis part cannot be placed in direct fluid communication with the patient prior to mixing. This can provide an added level of safety with respect to the preparation and administration of the ready-to-use formulation of the present disclosure as the single solution that cannot be placed in direct fluid communication with the patient physically cannot be fed to the patient unless it is first mixed with the other component. In this regard, if, by chance, the single solution part that physically cannot be placed in direct fluid communication with the patient were to have an undesirable concentration of constituents, such as potassium, sodium or the like, this configuration would necessarily ensure that the undesirable level of constituents is not fed or administered to the patient.

It should be appreciated that the separate dialysis parts of a multi-part dialysis solution can be housed or contained in any suitable manner such that the individual dialysis parts can be effectively prepared and administered. A variety of containers can be used to house the two parts, such as separate containers (e.g., flasks or bags) that are connected by a suitable fluid communication mechanism. The two or more separate dialysis parts can be separately sterilized and stored.

The dialysis solutions can comprise one or more suitable dialysis components (e.g. ingredients or constituents of a dialysis solution) such as osmotic agents, buffers, electrolytes or combination thereof. A variety of different and suitable acidic and/or basic agents can also be utilized to adjust the pH of the osmotic, buffer and/or electrolyte solutions or concentrates. For example, a variety of inorganic acids and bases can be utilized including hydrochloric acid, sulfuric acid, nitric acid, hydrogen bromide, hydrogen iodide, sodium hydroxide, the like or combination thereof.

Examples of osmotic agents include glucose, fructose, glucose polymers (e.g. maltodextrin, icodextrin, trehalose, cyclodextrins), glucose polymer derivatives (e.g. hydroxyethyl starch, modified starch), polyols, amino acids, peptides, proteins, amino sugars, N-acetyl glucosamine (NAG), glycerol and/or the like and combinations thereof. Examples of the buffers include bicarbonate, lactic acid/lactate, pyruvic acid/pyruvate, acetic acid/acetate, citric acid/citrate, amino acids, peptides, an intermediate of the KREBS cycle and/or the like and combinations thereof.

Examples of electrolytes include calcium, magnesium, sodium, potassium, chloride and/or the like and combinations thereof. For example, the dialysis solutions can comprise one or more electrolytes in the following ranges from: about 100 to about 140 mEq/L of Na⁺, about 70 to about 130 mEq/L of Cl⁻, 0.1 to about 4.0 mEq/L of Ca²⁺, 0.1 to about 4.0 mEq/L of Mg²⁺ and/or 0.1 to about 4.0 mEq/L of K⁺.

The dialysis solutions can preferably contain a dialysis component such as an osmotic agent to maintain the osmotic pressure of the solution greater than the physiological osmotic pressure (e.g. greater than about 285 mOsmol/kg). For example, glucose is the most commonly used osmotic agent because it provides rapid ultrafiltration rates. Other suitable types of osmotic agents can be used in addition to or as a substitute for glucose.

Another family of compounds capable of serving as osmotic agents in peritoneal dialysis solutions is that of glucose polymers or their derivatives, such as icodextrin, maltodextrins, hydroxyethyl starch, and the like. While these compounds are suitable for use as osmotic agents, they can be sensitive to low and high pH, especially during sterilization and long-term storage. Glucose polymers, such as icodextrin, can be used in addition to or in place of glucose in peritoneal dialysis solutions. In general, icodextrin is a polymer of glucose derived from the hydrolysis of corn starch. It has a molecular weight of 12-20,000 Daltons. The majority of glucose molecules in icodextrin are linearly linked with a (1-4) glucosidic bonds (>90%) while a small fraction (<10%) is linked by α (1-6) bonds.

The dialysis solutions or components can also comprise buffering agents such as bicarbonates and acids. The bicarbonates can comprise an alkaline solution such that the bicarbonate can remain stable without the use of a gas barrier overpouch or the like. The individual bicarbonate solution can have a pH that ranges above about 8.6, preferably about 9. The pH of the bicarbonate solution part can be adjusted with any suitable type of ingredient, such as sodium hydroxide and/or the like. Illustrative examples of the bicarbonate solution of the present disclosure can be found in U.S. Pat. No. 6,309,673, entitled BICARBONATE-BASED SOLUTION IN TWO PARTS FOR PERITONEAL DIALYSIS OR SUBSTITUTION IN CONTINUOUS RENAL REPLACEMENT THERAPY, issued on Oct. 30, 2001, the disclosure of which is herein incorporated by reference.

The acids can comprise one or more physiological acceptable acids, such as lactic acid, pyruvic acid, acetic acid, citric acid, hydrochloric acid and the like. The acids can be in an individual solution having a pH that ranges from about 5 or less, about 4 or less, about 3 or less, about 2 or less, about 1 or less, and any other suitable acidic pH. The use of an organic acid, such as lactic acid, alone or in combination with another suitable acid, such as a suitable inorganic acid including hydrochloric acid, another suitable organic acid (e.g. lactic acid/lactate, pyruvic acid/pyruvate, acetic acid/acetate, citric acid/citrate) and the like in the acid solution can make the solution more physiologically tolerable.

As discussed previously, the dialysis solutions of the present disclosure can be used in a variety of suitable applications. For example, the dialysis solutions can be used during peritoneal dialysis, such as automated peritoneal dialysis, continuous ambulatory peritoneal dialysis, continuous flow peritoneal dialysis and the like. It should be appreciated that the present disclosure can be used in a variety of different and suitable dialysis therapies to treat kidney failure.

Although the present disclosure, in an embodiment, can be utilized in methods providing a dialysis therapy for patients having chronic kidney failure or disease, it should be appreciated that the present disclosure can be used for acute dialysis needs, for example, in an emergency room setting. Lastly, as one of skill in the art appreciates, the intermittent forms of therapy (e.g., hemofiltration, hemodialysis, peritoneal dialysis and hemodiafiltration) may be used in the in center, self/limited care as well as the home settings.

### EXAMPLES

By way of example and not limitation, the following examples are illustrative of various embodiments of the present disclosure.

### Example 1

By utilizing a highly sensitive PBMC test, a pro-inflammatory substance was determined to be present in a batch of contaminated dialysis solutions (causing sterile peritonitis) in comparison to a control batch (not causing sterile peritonitis). The source of the pro-inflammatory substance was likely icodextrin. Using a combination of analytical techniques, it was deduced that the pro-inflammatory substance was peptidoglycan or similar in structure.

### Initial PBMC IL-6 Assay

The PBMC IL-6 assay uses a quantitative method to measure the level of pro-inflammatory cytokines such as interleukin-6 (IL-6 ), interleukin-1 or TNF-α that are secreted from human peripheral blood mononuclear cells (PBMC) upon stimulation with a pro-inflammatory substance. The general testing steps are as follows:
PBMCs are isolated from preferably fresh human blood of healthy donors.
The test and control articles are incubated with the cells overnight.
The culture media are collected and the secreted IL-6 quantified using Enzyme-Linked InmunoSorbent Assay (ELISA) techniques.

The initial PBMC-secreted cytokines (IL-6 , IL-1β, and TNF-α) test was performed on a first contaminated (i.e. containing pro-inflammatory substances) batch of dialysis solutions (06K23G38) (EXTRANEAL® dialysis solution by Baxter International). The results showed that an elevated IL-6 response was observed after incubation with samples from the contaminated batch (06K23G38) compared to controls, but only in one of the 6 donor PBMCs. The blood for these experiments was not fresh, but supplied by the Swiss Red Cross.

Initial studies using fresh blood, from a total of 12 human blood donors, did not show any difference in IL-6 responses between samples from the contaminated dialysis solutions and negative control samples. It was then determined that the PBMC IL-6 assay may be modified to better understand the variables affecting the PBMC IL-6 assay in order to optimize the assay.

### Development of a High Sensitivity PBMC IL-6 Assay

Several key variables of the PBMC IL-6 assay were identified and changes made to optimize assay performance. One change involved increasing the cell concentration used in the assay. As illustrated in FIGS. 2A and 2B, increasing cell concentrations increases the IL-6 response using the PBMC IL-6 assay. FIG. 2A shows the IL-6 response of various dialysis solutions using a cell concentration of 4×10⁵ cells/mL. FIG. 2B shows the IL-6 response of various dialysis solutions using a cell concentration of 4×10⁶ cells/mL. The IL-6 response is substantially higher using a cell concentration of 4×10⁶ cells/mL in the PBMC IL-6 assay compared to that with a cell concentration of 4×10⁵ cells/mL. Essentially, an advantage of using a higher cell concentration is that the ratio of the amplitude of the signal for PG to lipopolysaccharide (LPS) (endotoxin) is higher with a higher cell count than it is for a lower cell count.

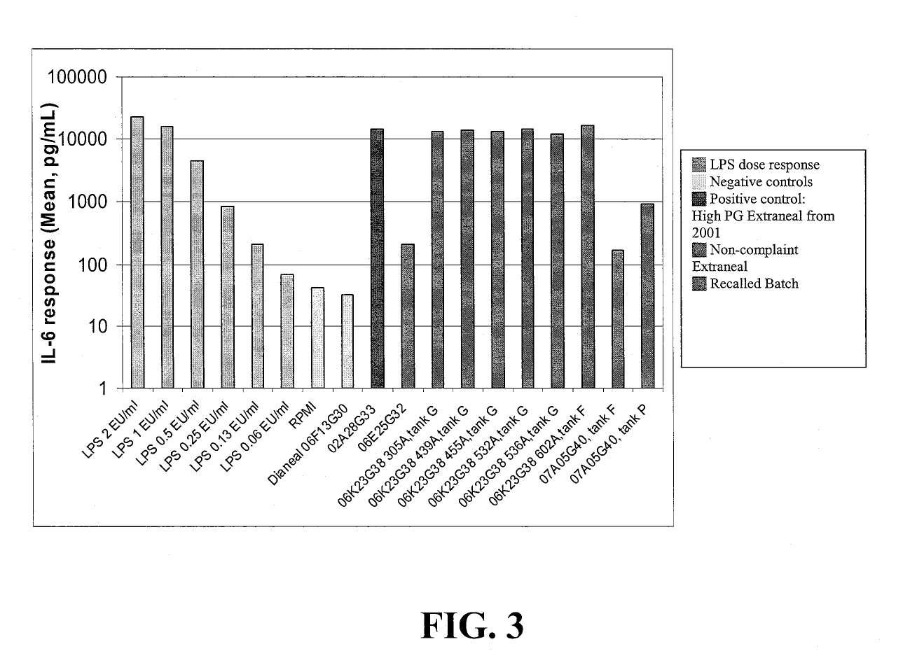

Another change involved excluding blood donors taking any medication with known anti-inflammatory properties. A third change involved using a high purity cell sample. These changes helped increase the sensitivity of the PBMC IL-6 assay and minimized spurious elevated responses with the controls. As illustrated in FIG. 3, by using the optimized procedures, the PBMC IL-6 assay was able to generate results showing a consistent elevation in IL-6 response from incubation of PBMC with samples from the first contaminated batch compared to negative controls and samples from an non-complaint batch of dialysis solutions (07A05G40).

Comparison with other Cell-Based Assays

During the development and use of the high sensitivity PBMC IL-6 assay, two other alternative cell-based assays were pursued in order to ascertain if different cell types could identify a biological response difference between a contaminated batch of dialysis solutions and an uncontaminated batch of dialysis solutions. The first of these alternative assays employed human peripheral blood mononuclear cells again using longer incubation times (up to 72 hours) in order to evaluate the activation of the T lymphocyte population present in the PBMC population. The second of the alternative assays evaluated the propensity of rat peritoneal mesothelial cells to detect a differential biological response between the contaminated batch and control batch of dialysis solutions. Neither of these alternative cell-based assays could detect any significant differences between the tested lots, confirming that using the high sensitivity PBMC IL-6 assay had an increased sensitivity limit to the pro-inflammatory substances.

Comparison with In Vivo Studies

In addition to the in vitro cell-based assays previously described, two studies were conducted using intraperitoneal injection of dialysis solutions into normal rats to ascertain if we could confirm that the contaminated batch could be differentiated from control batches in an in vivo model to strengthen the justification to use the in vitro PBMC assay.

### Rat Studies

Two rat studies were performed: 1) an acute, single infusion design, and 2) a chronic 7-day daily infusion design. In both designs, 4-hour dwell times were chosen, as significant re-absorption of fluid occurs during longer periods, such as in overnight dwells. Effluent was analyzed for both leukocyte counts and differentials, and effluent cytokine levels. No significant differences in cell counts or differentials were noted between the contaminated batch and control batch. No elevation in effluent pro-inflammatory cytokine levels was seen in the acute study. In contrast, a significant increase in the pro-inflammatory cytokines was seen in the 7-day exposure study in the first contaminated batch, in contrast to the control EXTRANEAL® batch and the control DIANEAL® batch (see Table 1).

TABLE 1
Cytokine Responses In The 7-Day Infusion Rat Model

| | | Result for Group | | | |
|---|---|---|---|---|---|
| | | Mean ± Std Err, (N), Significance | | | |
| | | First | | | High |
| | | Contaminated | Extraneal ® | Dianeal ® | Peptidoglyc. |
| Response | Time | Batch | Control | Control | Extraneal ® |
| Parameter | (hr) | Group 1 | Group 2 | Group 3 | Group 4 |
| Interleukin- | 4 | 120.5 ± 42.0 (8) | 17.5 ± 7.8 | 36.4 ± 23.4 | 116.4 ± 29.8 |
| 1 (pg/mL) | | | (8)^{aa} | (8)^{aa} | (10) |
| Interleukin- | 4 | 238.9 ± 144.4 | 52.4 ± 7.7 (8) | 78.0 ± 12.4 | 53.2 ± 2.8 (9) |
| 6 (pg/mL) | | (8) | | (8) | |
| Tumor | 4 | 76.5 ± 39.1 (8 | 0.4 ± 0.4 | 16.9 ± 16.8 | 26.5 ± 10.0 |
| Necrosis | | | (8)^{aaa} | (8)^{aaa} | (10) |
| Factor-alpha | | | | | |
| (pg/mL) | | | | | |
| Monocyte | 4 | 313.1 ± 155.5 | 24.7 ± 4.0 | 73.4 ± 38.0 | 173.0 ± 84.9 |
| Chemotactic | | (9) | (8)^{aaa} | (8)^{a} | (10) |
| Protein-1 | | | | | |
| (pg/mL) | | | | | |

Significantly Different Compared to Group 1:
^{a}= (p < 0.05);
^{aa}= (p < 0.01);
^{aaa}= (p < 0.001)

These results confirmed the existence of a pro-inflammatory signal in the contaminated batch, which was not sufficient enough to illicit an influx of leukocytes in this animal model. There was an elevation of all the monocyte derived pro-inflammatory cytokines tested: IL-1, TNF-α, MCP-1 and IL-6 , although the latter difference did not reach significance. It should be noted that the normal time course for maximum IL-6 response is much longer than for IL-1 and TNF-α, and thus higher and more consistent response would be expected if longer dwells had been possible. Nevertheless, the observed stimulation of monocytic cytokines in this study supported the continued use of the PBMC IL-6 assay.

### Glucose Polymers Studies

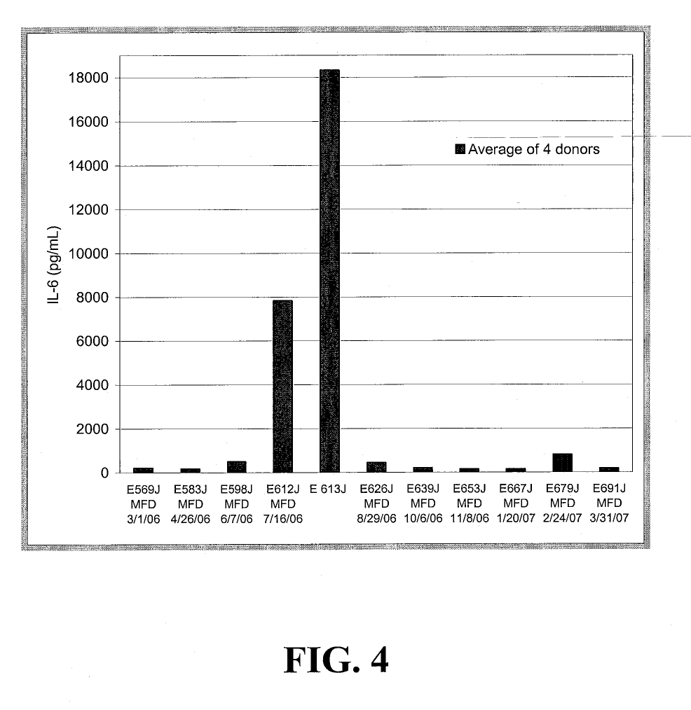

The glucose polymer icodextrin (E613J) as a raw material used to make the first contaminated dialysis solution batch was analyzed for IL-6 response along with several other selected icodextrin raw material lots. The icodextrin raw material was prepared at 7.5% of icodextrin in 0.9% saline (NaCl) and filtered via a 0.2 μm syringe filter prior to PBMC IL-6 assay. The PBMC IL-6 assay results (FIG. 4) indicate that there is a peak in the response in the icodextrin lot E613J.

Icodextrin samples from the other three icodextrin raw material lots (E608J, E609J, E611J) that were used in the second solution-mixing tank (tank F) were subsequently analyzed for PBMC-IL-6 response. These results (Table 2 below) confirm the similar apparent difference in PBMC-IL-6 responses with icodextrin raw material lots used in the preparation of a first contaminated EXTRANEAL® batch (06K23G38) in comparison to icodextrin raw material lots used in non-complaint EXTRANEAL® batches. Icodextrin raw material lots used to produce the first contaminated EXTRANEAL® batch express higher levels of IL-6 compared to icodextrin used in the non-complaint EXTRANEAL® batches.

TABLE 2
IL-6 Response (pg/mL) Of Icodextrin Raw Material

| Sample | MP009 | MP014 | MP019 | MP034 | Comments |
|---|---|---|---|---|---|
| RPMI + 2% | 59 | 643 | 802 | 249 | Negative control |
| plasma | | | | | |
| Dianeal ® | 818 | 176 | 142 | 170 | Negative control |
| 06F13G30 | | | | | |
| Extraneal ® | 3456 | 2233 | 158 | 164 | Negative Control |
| 06E25G32 | | | | | |
| Extraneal ® | 26314 | 17178 | 25279 | 14502 | First contaminated |
| 06K23G38 | | | | | batch |
| Icodextrin | 59 | 67 | 318 | 122 | Not used for first |
| E583J | | | | | contaminated batch |
| Icodextrin | 8906 | 1079 | 734 | 665 | Not used for first |
| E598J | | | | | contaminated batch |
| Icodextrin | 29577 | 9822 | 11978 | 14001 | Used for first |
| E608J | | | | | contaminated batch |
| Icodextrin | 26908 | 12961 | 16335 | 23806 | Used for first |
| E609J | | | | | contaminated batch |
| Icodextrin | 28260 | 9734 | 12784 | 12618 | Used for first |
| E611J | | | | | contaminated batch |
| Icodextrin | 36394 | 14425 | 21995 | 20525 | Used for first |
| E613J | | | | | contaminated batch |
| Icodextrin | 1586 | 974 | 732 | 2180 | Not used for first |
| E626J | | | | | contaminated batch |
| Icodextrin | 185 | 215 | 367 | 1223 | Not used for first |
| E639J | | | | | contaminated batch |

### Characterization of the IL-6 Inducing Substance

Subsequent efforts were directed to focus on the isolation, characterization and identification of the pro-inflammatory signal present in the first contaminated batch (06K23G38). A series of studies utilizing molecular weight cut off filters, ion-exchange columns, activated carbon treatment, and enzyme digestion, proved valuable in this respect and are described below:

The observation that the IL-6-inducing substance is likely to be peptidoglycan, or has a similar structure, prompted the evaluation of whether there was any correlation between IL-6 responses in icodextrin raw material and measurable peptidoglycan levels. When grouping peptidoglycan values into <0.74 ng/ml (the limit of detection (LOD)) for the silkworm larva plasma test), and those >0.74 ng/ml for the API series, a significant difference in IL-6 response was demonstrated using the PBMC IL-6 assay. The geometric mean ratios of >0.74 peptidoglycan to <0.74 ng/ml peptidoglycan for IL-6 values ranged from 20 to 100, and were highly statistically significant by donor and by combining donors.

Comparison of the Actual IL-6 Dose Response Curves from Two Contaminated Samples

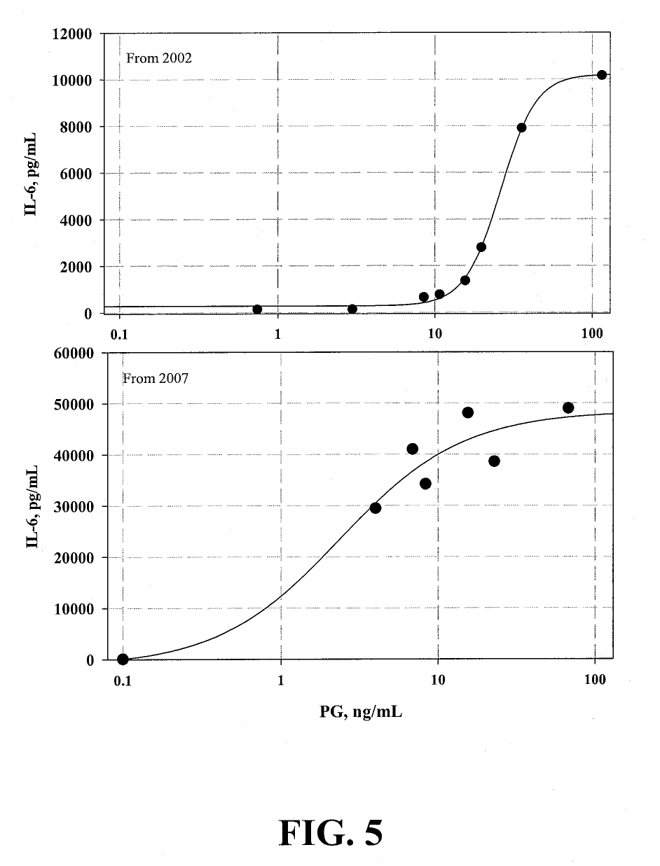

This study compares the actual IL-6 dose response curve of two contaminated batches of dialysis solution. One of the batches was a known contaminated batch (02A28G33)—EXTRANEAL® containing known peptidoglycan levels. The other batch was the contaminated batch (06K23G38A). A dose-response curve was obtained with the high sensitivity IL-6 assay using the known contaminated EXTRANEAL® sample as a reference standard. This contaminated sample had a high level of peptidoglycan (67.48 ng/ml) initially determined using the silkworm larva plasma test. It should be noted that the dose-response curve was created by performing serial dilutions of the contaminated solution with negative control EXTRANEAL® (e.g. uncontaminated). The undiluted solution was assayed at 67.48 ng/ml, while the rest of the other concentrations are the calculated concentrations derived from the dilution factor. The results are displayed in FIG. 5.

Two dose-response curves were made using the known contaminated batch as the reference standard. The first dose-response curve (2002) was made using a non-modified PBMC IL-6 assay. The second dose-response curve (2007) was made using a high sensitivity PBMC IL-6 assay. The dose-response curve for peptidoglycan and the corresponding IL-6 response using the non-modified PBMC IL-6 assay is different from that generated with the high sensitivity PBMC IL-6 assay (on the order of a 10-fold difference). Nevertheless, both PBMC IL-6 assays can be used to determine the corresponding peptidoglycan levels. However, the high sensitivity PBMC IL-6 assay can specifically be used to detect lower peptidoglycan levels.

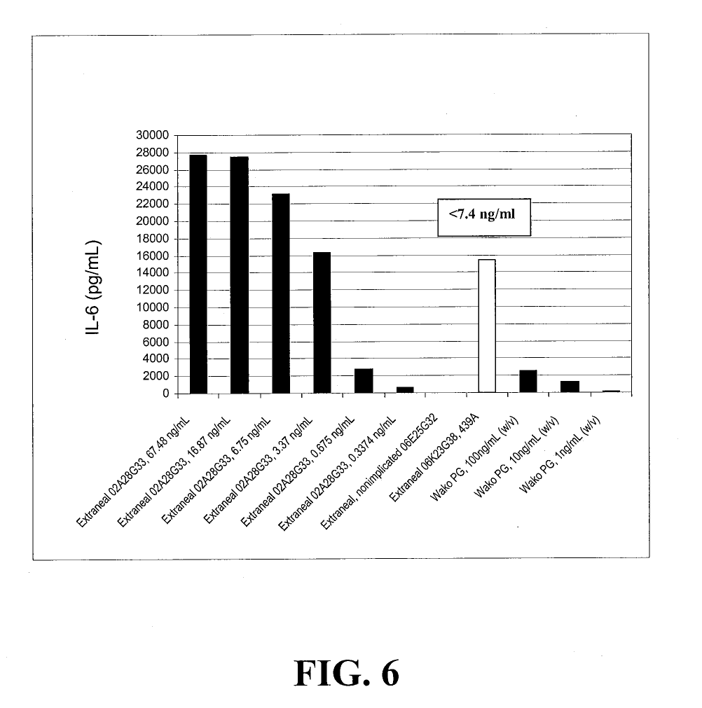

The IL-6 response from the contaminated batch (06K23G38A) was measured using the high sensitivity PBMC IL-6 assay. This IL-6 response is provided alongside the IL-6 responses obtained from the known contaminated EXTRANEAL® batch for comparison in FIG. 6. Although the actual peptidoglycan value for the contaminated batch (06K23G38) is only known to be <7.4 ng/ml, it should be appreciated that the IL-6 value corresponds to a peptidoglycan value from the dose-response curve (2007 of FIG. 5) that is also <7.4 ng/ml.

This study also shows that there appears to be no real differences in the peptidoglycan potency to induce IL-6 between the known contaminated EXTRANEAL® batch (02A28G33) and the contaminated batch (06K23G38). It should be noted that differences in the sensitivity of the non-modified PBMC IL-6 assay and the modified PBMC IL-6 assay (high sensitivity) should be considered when measuring the concentration of pro-inflammatory substance concentrations in different dialysis solutions.

### Example 2

This study applied statistics and mathematical models using two indices to data sets in attempt to reduce donor-to-donor variation observed in the PBMC IL-6 data.

A total of 4 replicates (4 wells per test solution) were set up for each test solution. Supernatants generated after overnight incubation of PBMCs with test solutions were analyzed for IL-6 concentration. IL-6 concentration was calculated using a working curve constructed using the mean of two chemiluminescence readings from each of seven IL-6 standard concentration levels. A non-linear regression was applied to determine the best theoretical fit so that each chemiluminescence reading from the test sample can be converted into IL-6 concentration. The response of a sample was determined and evaluated by either the mean IL-6 concentrations or the geometric median based on the 4 replicates. The geometric median is determined by the anti-log of the average of two middle log PBMC IL-6 values from the 4 replicates.

With a specific donor, a LPS (lipopolysaccharide)-normalized index is determined for each sample using an endotoxin equivalent unit or value based on the IL-6 responses to LPS standards at 6 concentration levels in each experiment. The mathematic model described this working curve is described below:

Log(IL-6)={min+max*exp[a+b*log(LPS)]}/{1+exp[a+b*log(LPS)]

where min, max, a, and b are the four parameters to be estimated by the SAS procedure NLIN.

The IL-6 values of each of the tested solutions were converted into the LPS normalized index using the following formula:

LPS Normalized Index=exp{{log[(log(IL-6)−min)/[max−(log(IL6)]−a}/b.

This index is simply expressed as the endotoxin equivalent unit for a donor for each tested solution including contaminated EXTRANEAL® dialysis solution samples, non-contaminated EXTRANEAL® samples and negative control samples.

Another index (i.e. % of positive) was also developed using the relative difference in percentage of the response from the positive control sample after subtracting the response from the negative control sample per donor.

% Positive=(x−Negative)/(Positive−Negative)*100%

where×is the log IL-6 value for sample, negative is the log IL-6 value for negative control, and positive is the log IL-6 value for the positive control.

The reason behind this approach is that the PBMC IL-6 response to a sample appears to be greatly donor dependent. Using the LPS normalized index based on individual donor's IL-6 response to LPS and relative difference as determined by % positive greatly diminishes the effect of the donor-to-donor variability.

The two indices (LPS-normalized index and the % positive index) were used in attempt to reduce donor-to-donor variability as a result of observation that IL-6 measurements are highly variable from donor-to-donor, and IL-6 response themselves could not be used to discriminate between positive and negative controls and between implicated and non-implicated samples. Instead, a relative elevation in IL-6 response has to be used to evaluate PBMC-IL-6 data.

Results from the two indices (LPS-normalized index and % positive index) have been determined based on either the average or geometric median of the four IL-6 measurements per donor. In addition, it appears that the % positive index is able to reduce to a greater extent the donor-to-donor variability compared to the LPS-normalized index or IL-6 concentration.

Utilizing results of the two indices, one can plot all samples tested from each of the 5 groups (DIANEAL® dialysis solution, Negative control RPMI±2% plasma, Positive control EXTRANEAL® dialysis solution—batch 02A28G33, implicated (contaminated) and non-implicated (non-contaminated) EXTRANEAL® dialysis solution) by PBMCs from 11 donors (see FIG. 7). It appeared that the assay can clearly discriminate among groups based on one of the two indices or both combined. The lowest values determined for the implicated samples appeared to be around 0.125 EU/mL LPS for the LPS normalized index and 50% for the % positive index (the solid lines in FIG. 6). All data points appeared to be clustered in lower left for majority of the negative and non-implicated samples and upper right corners for all implicated samples and positive control. There were a few “false positives” from the plots as indicated by observation of the non-implicated samples being clustered in the upper right corner, along with the implicated samples (FIG. 7).

A total of 33 data points were generated based on three non-implicated lots across 11 donors. There appeared to be less false positive based on results obtained for the non-implicated samples (FIG. 6) as indicated in the upper right corner when the geometric median is used compared to that when a mean is used. This suggested that the assay has improved capability to discriminate among groups when a geometric median is used. Furthermore, one can use the % positive index at 50% alone without increasing the number of “false” positive results observed from the non-implicated samples. In other word, adding the LPS normalized index at 0.125 does not increase the discriminating power of the assay when geometric median is used.

For all implicated samples tested, a cut-off value at a % positive index at 50% resulted in a 100% rate without any false negative results from the 66 data points generated from test of all implicated samples by 11 donors.

### Example 3

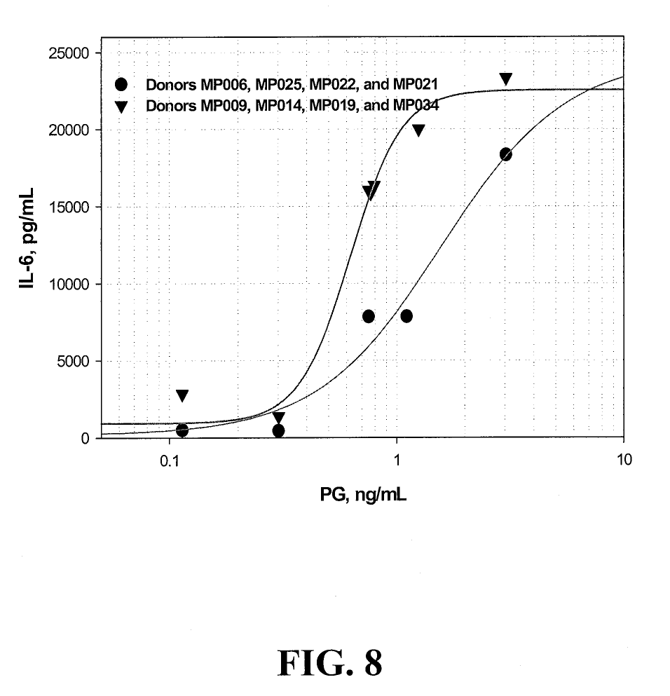

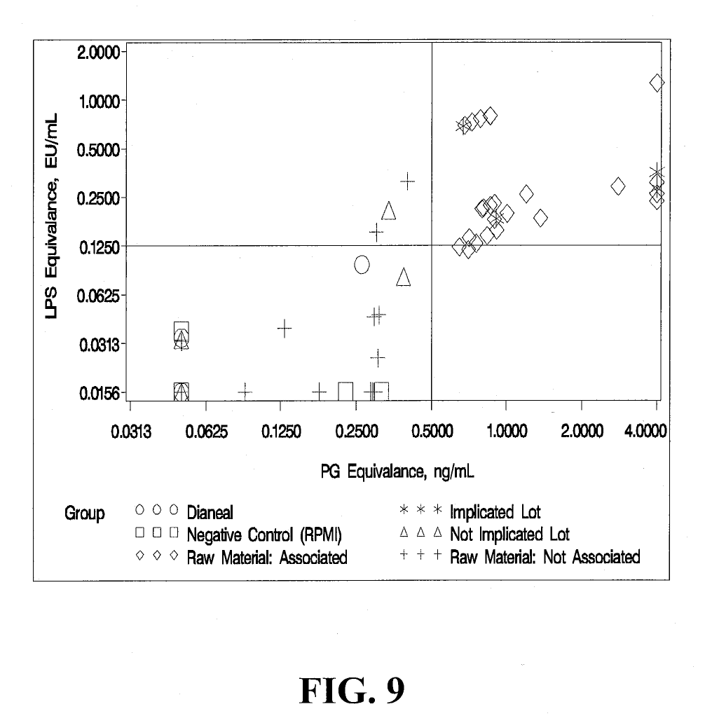

The purpose of this study was to analyze PBMC IL-6 and peptidoglycan (PG) results from the testing of several relevant icodextrin raw material lots to determine if there is a correlation between IL-6 response and PG level in those lots of icodextrin raw material. FIG. 8 shows a graph constructed using the mean IL-6 response of 4 donors against the PG values determined for each of the icodextrin samples. Best fit was estimated by use of a non-linear regression model, by fitting the experimental data points (symbols) to a sigmoid mathematical function (S-curve), a commonly observed dose-dependent biological response for many stimuli.

Using the relationships between IL-6 and LPS, as well as between IL-6 and PG, a LPS and PG equivalent value was determined for each donor sample and plotted in FIG. 8. Clearly samples of icodextrin raw material associated with the EXTRANEAL® dialysis solution batch (06K23G38) is clustered in the upper right corner. An apparent cutoff value appeared to be 0.125 EU/mL for LPS equivalence or 0.5 ng/mL of PG. The analysis results have shown strong evidence for a correlation between the PBMC IL-6 response and PG concentration in samples of icodextrin raw material.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for measuring a pro-inflammatory substance in a dialysis solution, the method comprising:
determining an IL-6 response versus a pro-inflammatory substance concentration of a reference standard containing the pro-inflammatory substance using a high sensitivity PBMC IL-6 assay, wherein a cell concentration of 2 million cells/mL or higher is used in the high sensitivity PBMC IL-6 assay and wherein all cells used in the high sensitivity PBMC IL-6 assay are from blood donors not taking any medication with known anti-inflammatory properties;
establishing a dose-response curve of the IL-6 response as a function of at least two different pro-inflammatory substance concentrations of the reference standard;
determining an IL-6 response of the dialysis solution using the high sensitivity PBMC IL-6 assay; and
calculating the corresponding pro-inflammatory substance concentration of the dialysis solution using the dose-response curve.

2-3. (canceled)

4. The method of claim 1, wherein the dose-response curve of the IL-6 response is established as a function of at least three different pro-inflammatory substance concentrations of the reference standard.

5. The method of claim 4, wherein the at least three different pro-inflammatory substance concentrations cover a pro-inflammatory substance range of at least two orders of magnitude.

6. The method of claim 4, wherein the at least three different pro-inflammatory substance concentrations cover a pro-inflammatory substance range of at least three orders of magnitude.

7. The method of claim 4, wherein the at least three different pro-inflammatory substance concentrations cover a pro-inflammatory substance range of at least four orders of magnitude.

8. The method of claim 1, wherein the dialysis solution comprises an osmotic agent selected from the group consisting of glucose polymers, glucose polymer derivatives and combinations thereof.

9. A method for measuring peptidoglycan in a dialysis solution, the method comprising:
determining an IL-6 response versus a peptidoglycan concentration of a reference standard containing the peptidoglycan using a high sensitivity PBMC IL-6 assay, wherein a cell concentration of 2 million cells/mL or higher is used in the high sensitivity PBMC IL-6 assay and wherein all cells used in the high sensitivity PBMC IL-6 assay are from blood donors not taking any medication with known anti-inflammatory properties;
establishing a dose-response curve of the IL-6 response as a function of at least two different peptidoglycan concentrations of the reference standard;
determining an IL-6 response of the dialysis solution using the high sensitivity PBMC IL-6 assay; and
calculating the corresponding peptidoglycan concentration of the dialysis solution using the dose-response curve.

10-11. (canceled)

12. The method of claim 9, wherein the dose-response curve of the IL-6 response is established as a function of at least seven different peptidoglycan concentrations of the reference standard.

13. The method of claim 12, wherein the at least seven different peptidoglycan concentrations cover a peptidoglycan range of at least two orders of magnitude.

14. The method of claim 12, wherein the at least seven different peptidoglycan concentrations cover a peptidoglycan range of at least three orders of magnitude.

15. The method of claim 12, wherein the at least seven different peptidoglycan concentrations cover a peptidoglycan range of at least four orders of magnitude.

16. The method of claim 9, wherein the dialysis solution comprises a glucose polymer as an osmotic agent.

17. The method of claim 16, wherein the glucose polymer comprises icodextrin.

18. A method for measuring a pro-inflammatory substance in a glucose polymer or a glucose polymer derivative, the method comprising:
determining an IL-6 response versus a pro-inflammatory substance concentration of a reference standard containing the pro-inflammatory substance using a high sensitivity PBMC IL-6 assay, wherein a cell concentration of 2 million cells/mL or higher is used in the high sensitivity PBMC IL-6 assay and wherein all cells used in the high sensitivity PBMC IL-6 assay are from blood donors not taking any medication with known anti-inflammatory properties;
establishing a dose-response curve of the IL-6 response as a function of at least two different pro-inflammatory substance concentrations of the reference standard;
determining an IL-6 response of the glucose polymer or glucose polymer derivative using the high sensitivity PBMC IL-6 assay; and
calculating the corresponding pro-inflammatory substance concentration of the glucose polymer or glucose polymer derivative using the dose-response curve.

19. The method of claim 18, wherein the glucose polymer comprises icodextrin.

20. A method for manufacturing a dialysis solution, the method comprising:
providing at least one dialysis component selected from the group consisting of glucose, glucose polymers and combinations thereof;
measuring the pro-inflammatory substance concentration of the dialysis component using a high sensitivity PBMC IL-6 assay; and
preparing a dialysis solution using the dialysis component if the pro-inflammatory substance concentration of the dialysis component is below a threshold concentration.

21. The method of claim 20, wherein the glucose polymer is icodextrin.

22. A method of providing dialysis to a patient, the method comprising:
determining an IL-6 response versus a pro-inflammatory substance concentration of a reference standard containing the pro-inflammatory substance using a high sensitivity PBMC IL-6 assay;
establishing a dose-response curve of the IL-6 response as a function of at least two different pro-inflammatory substance concentrations of the reference standard;
determining an IL-6 response of the dialysis solution using the high sensitivity PBMC IL-6 assay,
calculating the corresponding pro-inflammatory substance concentration of the dialysis solution using the dose-response curve; and
administering the dialysis solution to the patient.

23. The method of claim 22, wherein the dialysis solution is administered to the patient only if the pro-inflammatory substance concentration of the dialysis solution is below a threshold level.
